SEQUENCE LISTING (10 sequences)

SEQ ID NO: 1 - protein, 3 aa, Artificial sequence
  Synthetic construct
  MISC_FEATURE (1)..(1): Palmitoyl attached.
GHK

SEQ ID NO: 2 - protein, 4 aa, Artificial sequence
  Synthetic construct
  MISC_FEATURE (1)..(1): Palmitoyl attached.
GQPR

SEQ ID NO: 3 - protein, 5 aa, Artificial sequence
  Synthetic construct
  MISC_FEATURE (1)..(1): Palmitoyl attached.
KTTKS

SEQ ID NO: 4 - protein, 2 aa, Artificial sequence
  Synthetic construct
VW

SEQ ID NO: 5 - protein, 6 aa, Artificial sequence
  Synthetic construct
  MISC_FEATURE (1)..(1): Palmitoyl attached.
VGVAPG

SEQ ID NO: 6 - protein, 3 aa, Artificial sequence
  Synthetic construct
GHK

SEQ ID NO: 7 - protein, 3 aa, Artificial sequence
  Synthetic construct
KVK

SEQ ID NO: 8 - protein, 6 aa, Artificial sequence
  Synthetic construct
GPQGPQ

SEQ ID NO: 9 - protein, 15 aa, Artificial sequence
  Synthetic construct
RDFTKATNIRLRFLR

SEQ ID NO: 10 - protein, 6 aa, Artificial sequence
  Synthetic construct
KLDAPT

(19) United States
(12) Patent Application Publication
(10) Pub. No.: US 2007/0237735 A1
(43) Pub. Date: Oct. 11, 2007

(54) Anti-aging composition, kit and method of use

(75) Inventor: Sylvie Denommee, Le Gardeur (CA)

Correspondence Address: GOUDREAU GAGE DUBUC, 2000 MCGILL COLLEGE, SUITE 2200, MONTREAL, QC, H3A 3H3, CA

(73) Assignee: LABORATOIRES DERMO-COSMETIK INC.

(21) Appl. No.: 11/393,811

(22) Filed: Mar. 31, 2006

(51) Int. Cl. A61K 38/08; A61K 38/06; A61K 31/203; A61K 8/64

(52) U.S. Cl. 424/70.14; 514/17; 514/18; 514/16; 514/559

(57) ABSTRACT

An anti-aging composition comprising: (i) at least one biomimetic oligopeptide having a sequence of 20 amino acids or less; (ii) at least one lipoaminoacid; (iii) at least one pentacyclic triterpenoid selected from the group consisting of asiaticoside, madecassic acid, asiatic acid and madecassoside; (iv) at least one antioxidant; and (v) tetrahydropiperine; a kit comprising such composition and a method of use.

### CROSS REFERENCE TO RELATED APPLICATIONS

N/A.

### FIELD OF THE INVENTION

The present invention relates to compositions for topical application to human skin, and to methods of use and kits thereof. More specifically, the present invention relates to compositions for use in promoting the repair of damaged skin, essentially to reduce and/or prevent the age-related skin symptoms and impact of aggressive environmental influences.

### BACKGROUND OF THE INVENTION

In our youth-, aesthetic- and fashion-oriented society, skin appearance has become an ever larger public concern. The image of a beautiful and healthy looking skin is often associated with the absence of imperfections, such as wrinkles, and other signs of skin aging.

Chronological aging and biochemical changes (including hormonal changes) within the skin may result in a number of age-related symptoms such as thinning of the skin, appearance of fine lines, superficial and deep wrinkles, atrophy, loss of elasticity, collagen degeneration, loss of firmness, loss of tightness, loss of recoil from deformation, increased sagging, hyperkeratosis, discoloration and mottled pigmentation, coarse surface texture, roughness, decrease in skin cell proliferation rate, reduced skin ability to retain moisture.

In addition to the natural intrinsic aging process, a number of environmental aspects such as ultraviolet (UV) light, stress, excessive light or sun exposure, air conditioning, extreme temperatures, low humidity, wind, pollution, tobacco smoke, abrasives, harsh surfactants, are aggressive to the skin and contribute to the appearance of age-related skin symptoms.

Numerous efforts are constantly put into the science of cosmetology to develop cosmetic formulations with beneficial effects in reducing or preventing the symptoms of skin aging. The effects sought by the consumers of cosmetic care products are immediate, short, medium and long term.

Besides performance in reducing signs of skin aging itself, a number of additional issues underlie the development of a successful anti-aging cosmetic formulation, such as those of skin tolerance, pleasant odor, visually appealing and pleasant feeling texture and shelf life or stability.

A few examples of known products designed to improve skin condition are presented in the following documents:

| Publication Number | Title | Publication date |
|---|---|---|
| U.S. Pat. No. 5,939,082 | Methods of regulating skin appearance | 1999 Aug. 17 |
| | with vitamin B₃ compound | |
| U.S. Pat. No. 6,361,804 | Cosmetic formulations containing | 2002 Mar. 26 |
| | extracts from Phyllanthus Emblica and | |
| | Centella Asiatica and/or Bacopa | |
| | Monnieri | |
| U.S. Pat. No. 2005/089500A1 | Skin care composition | 2005 Apr. 28 |
| U.S. Pat. No. 2006/045896A1 | Topical compositions comprising | 2006 Mar. 02 |
| | benfotiamine and pyridoxine | |
| WO 04062678A1 | Method for preparing a Centella Asiatica | 2004 Jul. 29 |
| | extract rich in madecassoside and in | |
| | terminoloside | |
| WO 9739734A1 | Methods of regulating skin condition with | 1997 Oct. 30 |
| | Centella Asiatica extract | |
| WO 9834591A1 | Skin lightening compositions | 1998 Aug. 13 |

However, there still remains a need for new compositions for preventing, reducing or repairing age-related skin symptoms.

The present invention seeks to meet this and other needs.

The present description refers to a number of documents, the content of which is herein incorporated by reference in their entirety.

### SUMMARY OF THE INVENTION

The present invention is related to anti-aging compositions for use in the treatment or prevention of age-related skin symptoms.

More specifically, in accordance with the present invention, there is provided an anti-aging composition comprising at least one biomimetic oligopeptide having a sequence of 20 amino acids or less; at least one lipoaminoacid; at least one pentacyclic triterpenoid selected from the group consisting of asiaticoside, madecassic acid, asiatic acid and madecassoside; at least one antioxidant; and tetrahydropiperine.

In accordance with a particular embodiment of the present invention, there is also provided a kit comprising at least one biomimetic oligopeptide having a sequence of 20 amino acids or less, at least one lipoaminoacid, at least one pentacyclic triterpenoid selected from the group consisting of asiaticoside, madecassic acid, asiatic acid and madecassoside, at least one antioxidant, tetrahydropiperine and at least one cosmetically acceptable excipient in a commercial package together with instructions for preventing and/or reducing age-related skin symptoms.

In accordance with another particular embodiment of the present invention, there is also provided a method for reducing and/or preventing age-related skin symptoms, comprising topically applying a composition according to the present invention on human skin twice a day during at least 28 days.

In accordance with still another particular embodiment of the present invention, the present invention provides a composition comprising about 3% by weight of a mixture of Pal-GHK (SEQ ID No:1) and Pal-GQPR (SEQ ID No:2), about 1% by weight of dipalmitoyl hydroxyproline, about 0.1% by weight of a madecassoside extract having a purity of at least 95%, about 2% by weight Venuceane™, about 0.01% by weight Cosmoperine™.

In accordance with still another particular embodiment of the present invention, the present invention provides a composition comprising about 3% by weight of a mixture of Pal-GHK (SEQ ID No:1) and Pal-GQPR (SEQ ID No:2), about 1% by weight of dipalmitoyl hydroxyproline, about 0.1% by weight of a madecassoside extract having a purity of at least 95%, about 2% by weight Venuceane™, about 0.01% by weight Cosmoperine™, wherein said composition further comprises DC 1401, sepigel 305, DC 345, DC 9040, sepiplus 265, butylene glycol, phenonip, acety hexapeptide-3, saccharomyces/xylinum black tea ferment, hydrolysed soy protein, bambusa vulgaris extract, pisum sativum extract, glucosamine hydrochloride, xanthan gum, glucose, chondrus crispus, polydodecanamideaminium triazadiphenylethene sulfonate, polyvinylalcohol crosspolymer, prunus amygdalus dulcis seed extract, medicago sativa extract, laureth3, hydroxyethylcellulose, acetyl dipeptide-1 cetyl ester, NaOH 10% solution and water.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 is a diagram showing the results in roughness, firmness, tonicity, elasticity and hydration obtained in vivo in 28 days following twice-a-day topical application of a composition according to a particular embodiment of the present invention;

FIG. 2 is a set of three diagrams representing the reduction in the total area covered by wrinkles, as well as in length and depth of wrinkles on the forehead, obtained in vivo in 28 days following twice-a-day topical application of a composition according to a particular embodiment of the present invention;

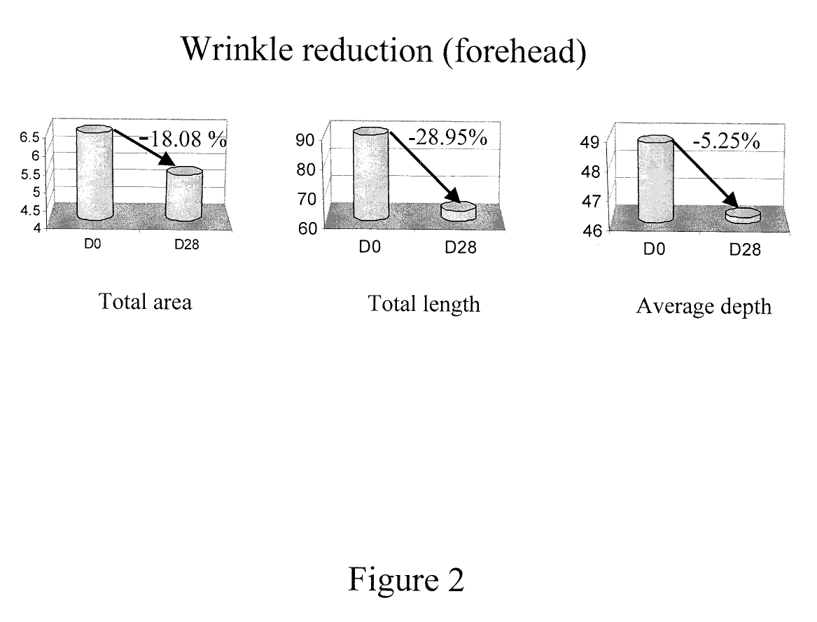

FIG. 3 is a set of three diagrams representing the reduction in the total area covered by wrinkles, as well as in length and depth of wrinkles on the crow's feet, obtained in vivo in 28 days following twice-a-day topical application of a composition according to a particular embodiment of the present invention; and

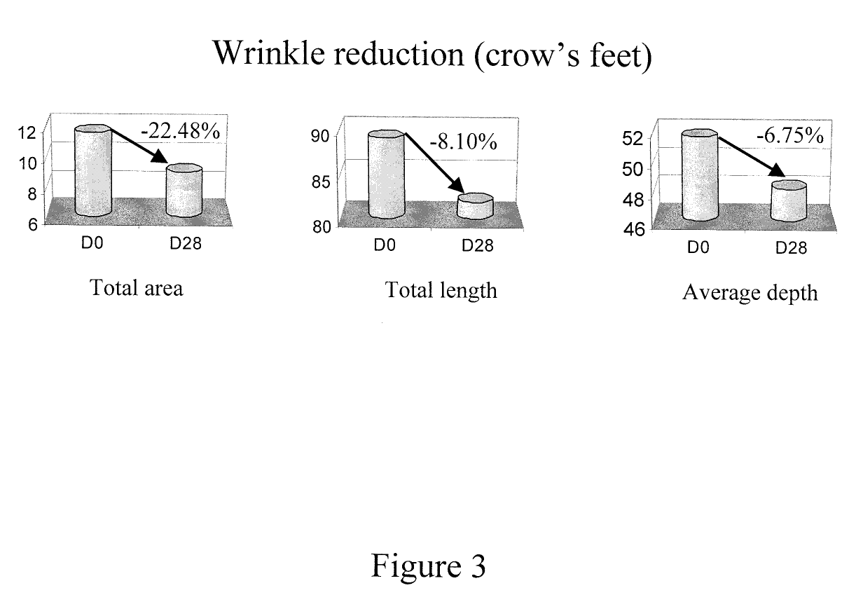

FIG. 4 is a set of three pictures showing the evolution at D0, D14 and D28 of the forehead skin aspect following twice-a-day topical application of a composition according to a particular embodiment of the present invention; and

FIG. 5 is a is a set of three pictures showing the evolution at D0, D14 and D28 of the crow's feet skin aspect following twice-a-day topical application of a composition according to a particular embodiment of the present invention.

### DESCRIPTION OF ILLUSTRATIVE EMBODIMENTS

The present invention relates to anti-aging compositions for use in the treatment or prevention of age-related skin symptoms, which is based on the combined action of distinct active ingredients in combination with tetrahydropiperine as a bio-availability enhancer.

The active ingredients and the bio-availability enhancer will be described in further details below.

Biomimetic oligopeptides

The biomimetic oligopeptides used in the compositions of the present invention are natural or synthetic oligopeptides having a sequence of 20 amino acids or less, which correspond to a fragment of a protein of the extracellular matrix in the dermis, such as α1-pro-collagen, α2-collagen 1, elastin, tropoelastin, fibronectin, laminin-5 or immunoglobulin IgG. Such oligopeptides are also known as matrikines, most of which are commercialized by Sederma, Vincience, Lipotec or Pentapharm.

Upon topical application, the biomimetic oligopeptides encompassed in the present invention act as messengers of a fake aggression on skin cells, thereby triggering activation of a mechanism similar to wound healing or matrix renewal. They indeed generally exercise feedback control on the process of connective tissue renewal and cell proliferation, for example by stimulating neo-synthesis of collagen I, III, IV, fibronectin, laminin-5 and/or glycosaminoglucans by fibroblasts.

Non-limiting examples of biomimetic oligopeptides used in the compositions of the present invention include: the tripeptide Palmitoyl-Glycyl-Histidyl-Lysine (SEQ ID No:1) (Pal-GHK, as Biopeptide-CL™ or in Maxi-lip™), the tetrapeptide Palmitoyl-Glycyl-Glutaminyl-Prolyl-Arginine (SEQ ID No:2) (Pal-GQPR or RIGIN™), the pentapeptide Palmitoyl-Lysyl-Threonyl-Threonyl-Lysyl-Serine (SEQ ID No:3) (Pal-KTTKS or Matrixyl™), a mixture of Pal-GHK and Pal-GQPR (Matrixyl™ 3000), a mixture of hesperidin methyl chalcone, dipeptide-2 (SEQ ID No:4) and Pal-GQPR (EYELISS™), a mixture of hexapeptide Palmitoyl-Val-Gly-Val-Ala-Pro-Gly (or Pal-VGVAPG) (SEQ ID No:5) with ceramide-2 (Dermaxyl™), hexapeptide-10 (an adhesion sequence from laminin, sold under the tradename Serilesine™), a mixture of GHK peptide (SEQ ID No:6) and wheat and soy protein hydrolysates (Aldenine™), palmitoyl tripeptide 3 (SEQ ID No:7) (Syn™-coll), hexapeptide-9 (SEQ ID No:8) (Collaxyl™), pentadeca-peptide-1 (SEQ ID No:9) (VINCI 01 Peptide) and hexapeptide-3 (SEQ ID No:10) (VINCI 02 Peptide).

The mimetic oligopeptides can be used in the compositions of the present invention in a concentration ranging between about 0.5 and about 10% by weight.

In specific embodiments, the above-listed peptides are included in the compositions according to the present invention in the concentration ranges listed in Table 1 below.

TABLE 1

| Biomimetic oligopeptide | Preferred concentration range |
|---|---|
| (Commercial name) | (% by weight) |
| Biopeptide-CL ™ or Maxi-lip ™ | 1-3 |
| RIGIN ™ | 1-3 |
| Matrixyl ™ | 1-3 |
| Matrixyl ™ 3000 | 1-3 |
| EYELISS ™ | 1-3 |
| Dermaxyl ™ | 0.5-2 |
| Serilesine ™ | 1-10 |
| Aldenine ™ | 2-5 |
| Syn ™-coll | 1-3 |
| Collaxyl ™ | 1-3 |
| VINCI 01 Peptide | 0.5-3 |
| VINCI 02 Peptide | 0.5-3 |

In a particular embodiment, more than one of the above-listed mimetic oligopeptides can be combined in the anti-aging compositions according to the present invention. In such case, the concentrations are cumulative.

Lipoaminoacids

A number of lipoaminoacids made from specific amino acids naturally present in the skin in general, or more specifically in the dermis, play an important role in the process of skin metabolism.

At least one lipoaminoacid is present in the compositions of the present invention, which has a structure similar or identical to a lipoaminoacid naturally present in the skin.

A non-limiting example of such lipoaminoacid is dipalmitoyl hydroxyproline (DPHP or SEPILIFT™ DPHP), a lipoaminoacid specific of the dermis. It results from a graft of a fatty acid (palmitic acid) onto a plant-derived amino acid (hydroxyproline).

DPHP has a protecting and firming action on the skin mainly by stimulating contraction of collagen fibers, stimulating the synthesis of laminin, rebalancing the cellular TIMPs/MMPs ratio (Tissue Inhibitors of Metalloproteinases/Metalloproteinases), inhibiting over-production of elastase enzymes, thereby protecting elastic fibers of the dermis, and reducing the level of free radicals.

DPHP may advantageously be used in a concentration ranging between about 0.5 and about 2% by weight of the compositions of the present invention.

Any other lipoaminoacid having an action similar or identical to that of DPHP may also be used in the compositions according to the present invention.

Pentacyclic triterpenoids

The pentacyclic triterpenoids used in the present invention include asiatic acid, madecassic acid, asiaticoside and/or madecassoside.

These active ingredients generally stimulate synthesis of collagen types 1 and 3 and promote the regeneration of the connective tissue. Madecassoside more specifically modulates epidermal inflammation reactions and can also participate in reducing the proliferation of keratinocytes, thereby protecting the cells against further inflammatory processes.

The pentacyclic triterpenoids encompassed by the present invention are advantageously extracted from Centella Asiatica. Centella Asiatica is a medicinal plant having wound healing and anti-inflammatory properties, which can be found in subtropical regions of the Indian Ocean. Its known active ingredients include such pentacyclic triterpenoids in the form of genins (asiatic and madecassic acids) and heterosides (asiaticoside and madecassoside).

Asiatic acid, madecassic acid, asiaticoside and/or madecassoside can be used in the compositions of the present invention alone or in combination.

Advantageously, the at least one pentacyclic triterpenoid is in highly purified or substantially pure form.

Pentacyclic triterpenoids are advantageously present in the compositions of the present invention in a concentration ranging between about 0.1 to 5%.

In a particular embodiment of the present invention, a titrated extract of asiaticoside, madecassic acid and asiatic acid (TECA-Titrated Extract of Centella Asiatica) is present in a concentration ranging between about 0.1 and about 1% by weight of the composition.

In another particular embodiment of the present invention, an extract of madecassoside having a purity of at least 75%, preferably 95% is present in a concentration ranging between about 0.1 and about 0.2% by weight of the composition.

Antioxidant

At least one antioxidant is present in the compositions of the present invention, non-limiting examples of which are Phyllanthus emblica (syn. Emblica officinalis, Emblica™, in a preferred concentration range of about 0.001-2%), Thermus Thermophyllus Ferment (Venuceane™, in a preferred concentration range of about 1-3%), Coenzyme Q10 (Lipoguard™, in a preferred concentration range of about 0.05-5%), alpha lipoic acid (or thioctic acid, ALA, vitamin N, in a preferred concentration range of about 0.05-1%), ascorbic acid (vitamin C, salts or esters thereof, such as palmitate, ascorbyl palmitate, magnesium ascorbyl palmitate, potassium ascorbyl tocopheryl phosphate, aminopropyl ascorbyl phosphate, trisodium ascorbyl palmitate phosphate, in a preferred concentration range of about 0.01-15%), alpha-tocopherol or derivatives thereof (acetate, nicotinate, linoleate, in a preferred concentration range of about 0.1-5%), nordihydroguaiaretic acid (NDGA, in a preferred concentration range of about 0.001-0.1%), superoxide dismutase like (SPD, in a preferred concentration range of about 0.5-2%), ethylbisiminoethylguaiacol manganese chloride (EUK 134, in a preferred concentration range of about 0.01-0.1%), a combination of hydroxypropyltrimonium maltodextrin crosspolymer and cucumis melo fruit extract (Extramel™ C, in a preferred concentration range of about 0.1-5%), a complex of lupin oil (White Lupin, Lupinus albus) and the active fraction of wheat germ oil (Triticum vulgare) (also known as alpha-lupaline in a preferred concentration range of about 0.05-5.0%), dipeptide decarboxy carnosine HCI (carcinine HCI, alistin, in a preferred concentration range of about 0.5-1.5%), L-carnosine (beta alanyl-I-histidine, dragosine, in a preferred concentration range of about 0.01-0.2%), a mixture of resveratol and viniferine (resveravine™, in a preferred concentration range of about 0.001-1.0%), betulinic acid in a preferred concentration range of about 0.05-0.5%), lycopene (lycosol, in a preferred concentration range of about 0.5-5%, a mixture of green tea (Camelia sinensis) and chrysanthellis (chrysanthellum indicum), (Lanatellis™, in a preferred concentration range of about 1-5%), ginko biloba extract (in a preferred concentration range of about 1-5%), mulberry extract (in a preferred concentration range of about 1-5%), idebenone in liposomial form (in a preferred concentration range of about 0.1-2%).

Tetrahydropiperine

Tetrahydropiperine is a pentanamide derivated from the piperine compound extracted from black pepper (Piper nigrum L.) or long pepper (Piper longum L.).

Tetrahydropiperine is advantageously added to the compositions of the present invention in a concentration ranging between about 0.0001 and about 0.1%, preferably about 0.01 to 0.1%, to optimize absorption of the active ingredients through the skin to the cellular targets of the dermis, thereby allowing an enhanced effective cellular action.

Further Optional Ingredients

The compositions according to the present invention optionally comprise further active ingredients of more immediate effect that help maximizing the response to anti-aging symptoms. Such very short term effect is often appreciated by consumers. Non-limiting examples of these further actives are as follows.

Acetyl-hexapeptide-3 (argireline™) and/or acetyl dipeptide-1 cetyl ester (calmosensine™) offer a myo-relaxing effect that reduces dermo-crispation, therefore allowing dermo-relaxation.

Vegetal and biotechnological extracts, such as soy oligopeptides (Ridulisse C™), which enhance metabolic activity of fibroblastes, a combination of bambusa vulgaris, pisum sativum and glucosamine (Dermox™ SRC), and medicago sativa (vitanol™), which assist skin metabolism and enhance skin surface renewal, black tea ferment extract, saccharomyces and xylinum, which act on adipocytes to remodel contours of skin suffering from loss of adipose tissue, and any other vegetal extract having similar or complementary actions.

Wheat protein hydrolysate, glycine soya protein and/or prunus amygdallus dulcis extract and/or synthetic polymer such as PVP crosspolymer offer an immediate skin tensing effect via the formation of a thin viscoelastic film on the skin (filmogen action).

Inert substances that act as light catchers or diffusers, often referred to as “soft focus effect pigments”, such as LipoLight™ OAP/PVP (polydodecanamideaminium triazadiphenylethene sulfonate, polyvinylalcohol crosspolymer), Ronasphere™ LDP (modified silica), and any other substance having a similar effect of visual masking of wrinkles.

The compositions according to the present invention may further comprise at least one cosmetically acceptable excipient of conventional type. The excipient(s) used in the composition depend on the final formulation desired.

Non-limiting examples of possible excipients to be included in the compositions according to the present invention are as follows: texturing agents, such as carbomer, acrylate derivatives (such as sepiplus™ 265, sepigel™ 305, simulgel™ NS, DC RM 2051), elastomer (such as DC9040, DC9509, DC9701, DC9041) and/or xanthan gum; emollients, such as silicones or vegetal oils; moisturizing agents; preservatives, fragrances, essential oils, and/or emulsioning agents of lamellar, anionic, cationic and/or non-ionic type, such agents allowing creation of water-in-oil or oil-in-water systems for example, as well as of microemulsion or nanoemulsion.

Without being so limited, a composition according to the present invention can be formulated as a cream, a gel, a lotion, a serum, a mask-type cream, an emulsion, a microemulsion, a nanoemulsion or any suitable formulation suitable for topical application.

The compositions according to the present invention may offer significant results in reducing age-related skin symptoms, more specifically in decreasing roughness, increasing firmness, elasticity, hydration and tonicity, as well as decreasing the total area covered by wrinkles, length and depth of the wrinkles.

Topical application of the compositions according to the present invention once or twice a day may provide a significant anti-aging effect within 28 days or less, although the treatment may be pursued after this 28-day period.

The compositions according to the present invention are well-tolerated on the skin in general and on the skin surrounding the eyes, which is particularly sensitive.

The present invention is illustrated in further details by the following non-limiting examples.

### EXAMPLE 1

### Anti-Aging Composition and Method of Preparation Thereof

A composition as set out in Table 2 below has been prepared and tested in vivo.

TABLE 2

| | | Concentration | |
|---|---|---|---|
| | | by weight of | |
| | | composition | Quantity |
| Step | Ingredient | (%) | (g) |
| A) | DC 1401 | 1.50 | 7.50 |
| | sepigel 305 | 1.50 | 7.50 |
| | DC 345 | 9.60 | 48.00 |
| | DC 9040 | 2.88 | 14.40 |
| | sepiplus 265 | 0.46 | 2.30 |
| B) | Water | 32.85 | 164.25 |
| | NaOH 10% solution | 0.60 | 3.00 |
| C) | Sepilift DPHP/from SEPPIC | 1.00 | 5.00 |
| | MADECASSOSIDE/from BAYER | 0.10 | 0.50 |
| | cosmoperine/from SABINSA | 0.01 | 0.05 |
| | Matrixyl 3000/from SEDERMA | 3.00 | 15.00 |
| | CRODA | | |
| | Venuceane/from SEDERMA | 2.00 | 10.00 |
| | CRODA | | |
| D) | BUTYLENE GLYCOL | 2.00 | 10.00 |
| | PHENONIP | 0.50 | 2.50 |
| E) | ACETYL HEXAPEPTIDE-3 | 10.00 | 50.00 |
| | SACCHAROMYCES/XYLINUM | 3.00 | 15.00 |
| | BLACK TEA FERMENT | | |
| | HYDROLYZED SOY PROTEIN | 4.00 | 20.00 |
| | BAMBUSA VULGARIS | 3.00 | 15.00 |
| | EXTRACT, PISUM SATIVUM | | |
| | (PEA) EXTRACT, GLUCOSAMINE | | |
| | HCL | | |
| | XANTHAN GUM, GLUCOSE, | 10.00 | 50.00 |
| | CHONDRUS CRISPUS | | |
| | (CARRAGEENAN) | | |
| | POLYDODECANAMIDEAMINIUM | 3.00 | 15.00 |
| | TRIAZADIPHENYLETHENE | | |
| | SULFONATE, | | |
| | POLYVINYLALCOHOL | | |
| | CROSSPOLYMER | | |
| | WATER, PRUNUS AMYGDALUS | 3.00 | 15.00 |
| | DULCIS (SWEET ALMOND) SEED | | |
| | EXTRACT | | |
| | MEDICAGO SATIVA (ALFALFA) | 4.00 | 20.00 |
| | EXTRACT | | |
| | BUTYLENE GLYCOL, WATER, | 2.00 | 10.00 |
| | LAURETH-3, | | |
| | HYDROXYETHYLCELLULOSE, | | |
| | ACETYL DIPEPTIDE-1 CETYL | | |
| | ESTER | | |
| | | 100.00 | 500.00 |

The above composition was prepared by first adding sodium hydroxide to purified water and agitating until its complete dissolution. The resulting solution was then heated to 65° C., and DPHP added under agitation. The agitation continued until obtaining an homogenous opalescent solution, to which Cosmoperine™ and TECA were added. The whole mixture was then allowed to cool down at room temperature and Matrixyl™ 3000 and Venuceane were added. Again, the solution was agitated to reach homogeneity.

The remaining ingredients of the composition were then added successively. Ingredient L39 is desirably added by sprinkling under agitation. The final pH of the resulting composition ranges between 5.5 and 6.5.

The formulation thus prepared is in the form of a serum.

### EXAMPLE 2

### Non-Invasive in vivo Study of the Anti-Aging Composition of Example 1

A double-blind study of 28 days was done on 82 female volunteers of all skin types and showing signs of aging skin. The average age of the participants was 50.1 years old.

Conditions of the Study:

A small quantity of the formulation of Example 1 was applied twice a day (morning and evening), on a perfectly clean skin, on the face and on the anterior surface of one forearm during 28 days, followed by a conventional skin care product appropriate to each skin type, as would normally happen in everyday life (so as not to distort the results).

At each visit, the volunteers should have a perfectly cleaned skin (more than 2 hours before a measure), with no skin care product applied on it. The visits were at D0, D14 (14^{th} day of application) and D28 (28^{th} and last day of application).

The measures were generally performed at a temperature of 20° C. with a relative humidity of 40-60%, under constant luminosity conditions.

Unless otherwise specified, the significance of the results is evaluated with the Student's test.

Quantitative Results of Non-Invasive Measures:

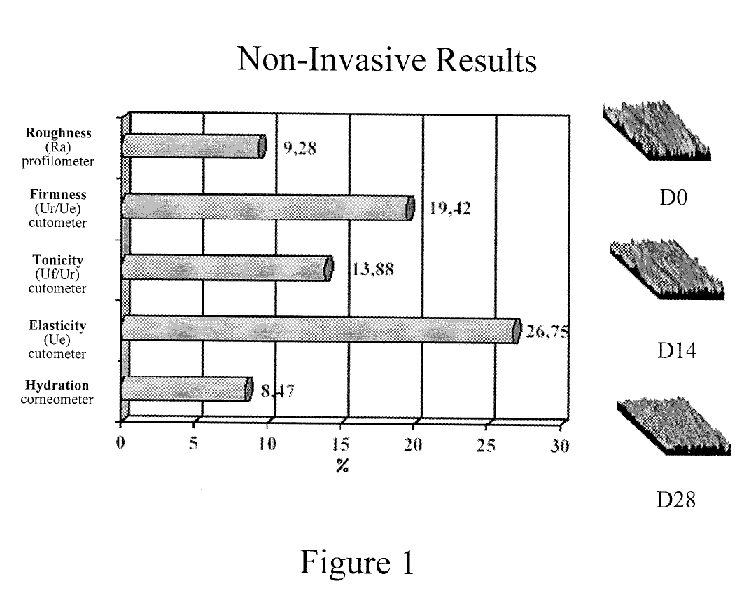

The results obtained are summarized in FIG. 1 and presented below in further detail.

Skin hydration was measured with a corneometer™ CM 825 at 4 points of the face (forehead, two different places on cheeks and chin) and on the forearms. Volunteers applied the formulation on the whole face and on the right forearm. The left forearm was thus used as control.

A corneometer measures skin hydration through its capacitance, i.e. an electrical property. The dry stratum corneum (outermost layer of the epidermis) indeed acts like a dielectric medium, and addition of water makes the stratum corneum responsive to an electrical field. The capacitance measurements involve two oppositely charged plates held in close proximity. An electric field is formed between them, and the maximum charge on each plate is known as the capacitance. When dielectric materials are introduced into the gap between the two plates, they increase this capacitance value.

The quantitative results of skin hydration in this example are shown in Table 3 below:

TABLE 3

| | | Left forearm | Right | |
|---|---|---|---|---|
| Skin hydration | Face | (control) | forearm | Delta control |
| D0 | 74.27 | 62.27 | 63.2 | N/A |
| D28 | 73.44 | 63.6 | 70 | N/A |
| Delta D28/D0 (%) | −1.11 | 2.14 | 10.76 | +8.62 |
| Significance | S | S | S | N/A |

When comparing both forearms, it can be noted that skin hydration increased during the evaluation period, which is a positive result for the composition tested. The slight decrease of skin hydration observed on the face could be attributable to the period of the test (late October/ November).

Elasticity, firmness and tonicity were measured with a cutometer™ SEM 575. Different elasticity are established and analyzed by the apparatus itself (Ur, Ue, Uf). The resulting values for these measures range between 0 and 1. The closer elasticity and firmness values are to 1, the better. The closer tonicity is to 0, the better. The exact results are presented in Table 4.

TABLE 4

| | Elasticity | Tonicity | Firmness |
|---|---|---|---|
| D0 | 0.4799 | 0.3858 | 0.4359 |
| D28 | 0.6082 | 0.3322 | 0.5206 |
| Delta D28/D0 (%) | 26.75 | −13.88 | 19.42 |
| Significance | S | S | S |

The values of elasticity, firmness and tonicity clearly improved during the 28-day evaluation period.

Profilometry was used to evaluate the effects of the composition of Example 1 in terms of both roughness and wrinkle reduction. Negative replicas (prints) made in silicon-type paste of skin relief at predetermined sites (for example forearm, forehead, crow's feet) were made for each volunteer, at each visit. These replica were then put under almost horizontal light, thereby generating a shadow behind each wrinkle. An image of the result, including the shadows, is captured by a CCD camera, and digitalized. Various softwares then analyze the data, in terms of roughness or wrinkle measure.

Roughness was measured from prints taken on the anterior surface of forearms. The lower the roughness, the better. The results are summarized in Table 5 below:

TABLE 5

| Roughness | Forearm |
|---|---|
| D0 | 45.10 |
| D28 | 40.91 |
| Delta D28/D0 (%) | −9.28 |
| Significance | S |

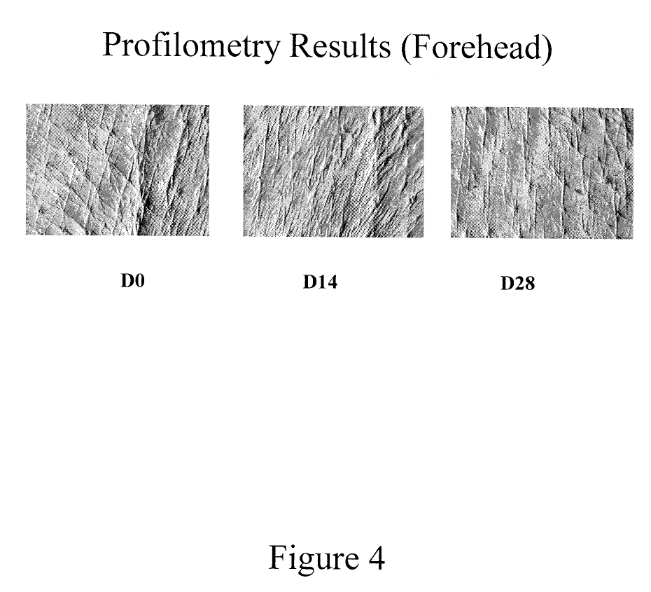

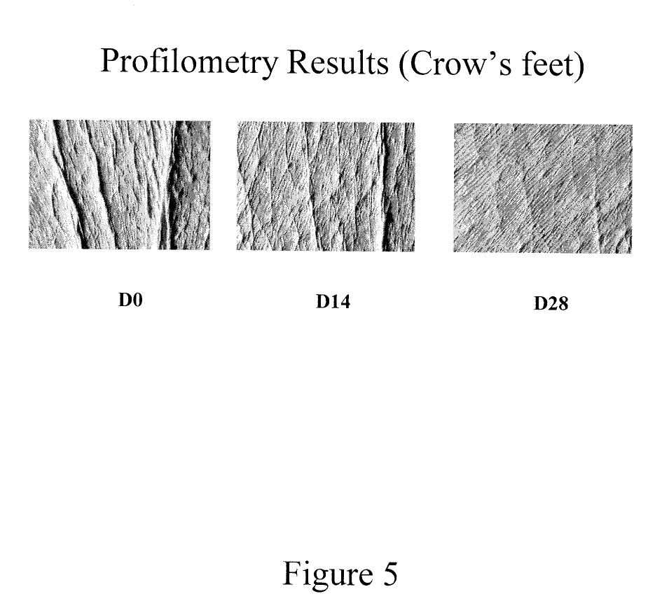

Dimension measures of wrinkle reduction on the forehead and crow's feet were also taken. The average and maximum results are presented in Tables 6 and 7 below, and illustrated in FIGS. 4 and 5.

TABLE 6

| | | Total | Average |
|---|---|---|---|
| Forehead | Total area | length | depth |
| D0 | 6.49 | 89.68 | 48.77 |
| D28 | 5.32 | 63.72 | 46.21 |
| Delta D28/D0 (%) | −18.08 | −28.95 | −5.25 |
| Significance | S | S | S |

TABLE 7

| | | Total | Average |
|---|---|---|---|
| Crow's feet | Total area | length | depth |
| D0 | 11.62 | 89.15 | 51.71 |
| D28 | 9.01 | 81.93 | 48.22 |
| Delta D28/D0 (%) | −22.48 | −8.10 | −6.75 |
| Significance | S | S | S |

On both the forehead and the crow's feet parts of the face, a significant reduction has been observed on all aspects evaluated by profilometry after 28 days of treatment. These quantitative results are very satisfactory for the composition of the present invention.

FIGS. 4 and 5 additionally show that marked surface wrinkles progressively disappear, in term of depth, size and number.

Although the present invention has been described hereinabove by way of specific embodiments thereof, it can be modified, without departing from the spirit and nature of the subject invention as defined in the appended claims.

What is claimed is:

1. An anti-aging composition comprising:
(i) at least one biomimetic oligopeptide having a sequence of 20 amino acids or less;
(ii) at least one lipoaminoacid;
(iii) at least one pentacyclic triterpenoid selected from the group consisting of asiaticoside, madecassic acid, asiatic acid and madecassoside;
(iv) at least one antioxidant; and
(v) tetrahydropiperine.

2. The composition of claim 1, wherein the at least one biomimetic oligopeptide is a natural or synthetic peptide corresponding to a fragment of a dermis extracellular protein, or mixture thereof.

3. The composition of claim 2, wherein the dermis extracellular protein is selected from the group comprising α1-pro-collagen, α2-collagen 1, elastin, tropoelastin, fibronectin, laminin-5 and immunoglobulin IgG.

4. The composition of claim 3, wherein said at least one biomimetic oligopeptide is selected from the group consisting of Pal-GHK (SEQ ID No:1), Pal-GQPR (SEQ ID No:2), Pal-KTTKS (SEQ ID No:3), a mixture of Pal-GHK and Pal-GQPR, a mixture of Hesperidin Methyl Chalcone, dipeptide-2 (SEQ ID No:4) and Pal-GQPR, a mixture of Pal-VGVAPG (SEQ ID No:5) with ceramide-2, hexapeptide-10 (Serilesine™), a mixture of GHK peptide (SEQ ID No:6) and wheat and soy protein hydrolysates, palmitoyl tripeptide 3 (SEQ ID No:7), hexapeptide-9 (SEQ ID No:8), pentadeca-peptide-1 (SEQ ID No:9), hexapeptide-3 (SEQ ID No:10).

5. The composition of claim 4, wherein said at least one biomimetic oligopeptide is present in a concentration ranging between about 0.5 and about 10% by weight.

6. The composition of claim 5, wherein said at least one biomimetic oligopeptide is selected from the group consisting of Pal-GHK (SEQ ID No:1), Pal-GQPR (SEQ ID No:2), Pal-KTTKS (SEQ ID No:3), and a mixture of Pal-GHK and Pal-GQPR, in a concentration ranging between about 1 and about 3% by weight.

7. The composition of claim 6, wherein said at least one biomimetic oligopeptide is a mixture of Pal-GHK (SEQ ID No:1) and Pal-GQPR (SEQ ID No:2) in a concentration ranging between about 1 and about 3% by weight.

8. The composition of claim 1, wherein said at least one lipoaminoacid is dipalmitoyl hydroxyproline.

9. The composition of claim 8, wherein dipalmitoyl hydroxyproline is present in a concentration ranging between about 0.5 and about 2% by weight.

10. The composition of claim 1, wherein said at least one pentacyclic triterpenoid is a Centella Asiatica extract.

11. The composition of claim 10, wherein said at least one pentacyclic triterpenoid is a titrated extract of asiaticoside, madecassic acid and asiatic acid (TECA) or a madecassoside extract having a purity of at least 75%.

12. The composition of claim 1 1, wherein said at least one pentacyclic triterpenoid is a madecassoside extract having a purity of at least 95%.

13. The composition of claim 12, wherein said at least one pentacyclic triterpenoid is present in a concentration ranging between about 0.1 and about 5% by weight.

14. The composition of claim 13, wherein said at least one pentacyclic triterpenoid is present in a concentration ranging between about 0.1 and about 0.5% by weight.

15. The composition of claim 12, wherein the madecassoside extract having a purity of at least 95% is present in a concentration ranging between about 0.1 and about 0.2% by weight.

16. The composition of claim 1, wherein the antioxidant is selected from the group consisting of Phyllanthus emblica (Emblica ), Thermus Thermophyllus Ferment (Venuceane™), Coenzyme Q10 (Lipoguard™), alpha lipoic acid, ascorbic acid (vitamin C, salts or esters thereof), alpha-tocopherol or derivatives thereof, nordihydroguaiaretic acid (NDGA), superoxide dismutase like (SPD), ethylbisiminoethylguaiacol manganese chloride (EUK 134), a combination of hydroxypropyltrimonium maltodextrin crosspolymer and cucumis melo fruit extract (Extramel™ C), a complex of lupin oil and an active fraction of wheat germ oil (alpha-lupaline), dipeptide decarboxy carnosine HCI (carcinine HCI), L-carnosine, a mixture of resveratol and viniferine (resveravine ™), betulinic acid, lycopene (lycosol), a mixture of green tea and chrysanthellis (lanatellis™), ginko biloba extract, mulberry extract and idebenone in liposomial form.

17. The composition of claim 16, wherein the antioxidant is Thermus Thermophyllus Ferment (Venuceane™).

18. The composition of claim 17, wherein the antioxidant is present in a concentration ranging between about 1 to about 3%.

19. The composition of claim 1, wherein tetrahydropiperine is present in a concentration ranging between 0.0001 and about 0.1% by weight.

20. The composition of claim 19, wherein tetrahydropiperine is present in a concentration ranging between about 0.01 and about 0.1% by weight.

21. An anti-aging composition according to claim 1 comprising about 3% by weight of a mixture of Pal-GHK (SEQ ID No:1) and Pal-GQPR (SEQ ID No:2), about 1% by weight of dipalmitoyl hydroxyproline, about 0.1% by weight of a madecassoside extract having a purity of at least 95%, about 2% by weight Venuceane™, about 0.01% by weight Cosmoperine™.

22. A composition according to claim 1, further comprising at least one cosmetically acceptable excipient.

23. A composition according to claim 1, further comprising at least one additional active ingredient.

24. An anti-aging composition according to claim 1 comprising about 3% by weight of a mixture of Pal-GHK (SEQ ID No:1) and Pal-GQPR (SEQ ID No:2), about 1% by weight of dipalmitoyl hydroxyproline, about 0.1% by weight of a madecassoside extract having a purity of at least 95%, about 2% by weight Venuceane™, about 0.01% by weight Cosmoperine , wherein said composition further comprises DC 1401, sepigel 305, DC 345, DC 9040, sepiplus 265, butylene glycol, phenonip, acetyl-hexapeptide-3, saccharomyces/xylinum black tea ferment, hydrolysed soy protein, bambusa vulgaris extract, pisum sativum extract, glucosamine hydrochloride, xanthan gum, glucose, chondrus crispus, polydodecanamideaminium triazadiphenylethene sulfonate, polyvinylalcohol crosspolymer, prunus amygdalus dulcis seed extract, medicago sativa extract, laureth-3, hydroxyethylcellulose, acetyl dipeptide-1 cetyl ester, NaOH 10% solution and water.

25. An anti-aging composition according to claim 24, comprising about 1.5% by weight DC 1401, about 1.5% by weight sepigel 305, about 10% by weight DC 345, about 3% by weight DC 9040, about 0.5% by weight sepiplus 265, about 2% by weight butylene glycol, about 0.5% by weight phenonipTM , about 10% by weight acetyl-hexapeptide-3, about 3% by weight saccharomyces/xylinum black tea ferment, about 4% by weight hydrolysed soy protein, about 3% by weight of a mixture of bambusa vulgaris extract, pisum sativum extract and glucosamine hydrochloride, about 10% by weight of a mixture of xanthan gum, glucose and chondrus crispus, about 3% by weight polydodecanamideaminium triazadiphenylethene sulfonate, polyvinylalcohol crosspolymer, about 3% by weight of prunus amygdalus dulcis seed extract in water, about 4% by weight medicago sativa extract, about 2% by weight of a mixture of butylene glycol, laureth-3, hydroxyethylcellulose and acetyl dipeptide-1 cetyl ester in water, about 0.6% by weight NaOH 10% solution, the balance being water.

26. The composition according to claim 1, formulated in a form of a cream or a mask-type cream.

27. The composition according to claim 1, formulated in a form of a lotion.

28. The composition according to claim 1, formulated in a form of an emulsion, a microemulsion or a nanoemulsion.

29. The composition according to claim 1, formulated in a form of a serum.

30. A method for preventing and/or reducing age-related skin symptoms, comprising topically applying a composition according to claim 1 on human skin twice a day during at least 28 days.

31. A kit comprising at least one biomimetic oligopeptide having a sequence of 20 amino acids or less, at least one lipoaminoacid, at least one pentacyclic triterpenoid selected from the group consisting of asiaticoside, madecassic acid, asiatic acid and madecassoside, at least one antioxidant, tetrahydropiperine and at least one cosmetically acceptable excipient in a commercial package together with instructions for preventing and/or reducing age-related skin symptoms.
